(19) United States
(12) Patent Application Publication
(10) Pub. No.: US 2009/0215894 A1
(43) Pub. Date: Aug. 27, 2009

(54) INHIBITION OF PHOSPHATASE ACTIVITY OF SOLUBLE EPOXIDE HYDROLASE AMINO TERMINUS AND USES THEREOF

(75) Inventors: Bruce D. Hammock, Davis, CA (US); Christophe Morisseau, West Sacramento, CA (US); John W. Newman, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 12/063,573

(22) PCT Filed: Aug. 14, 2006
(86) PCT No.: PCT/US06/31589

§ 371 Date: Apr. 22, 2009

Related U.S. Application Data
(60) Provisional application No. 60/708,107, filed on Aug. 12, 2005.

(51) Int. Cl. A61K 31/20; C12N 9/99; A61K 31/10; C07C 309/00

(52) U.S. Cl. 514/558; 435/184; 514/711; 562/109

(57) ABSTRACT

Inhibitors of the phosphatase activity of soluble epoxide hydrolase (sEH) are provided and are useful for in the treatment of diseases. These Inhibitors are based on derivatives of various epoxide hydrolase substrates that mimic the enzyme substrate so that there Is stable Interaction with the enzyme catalytic site. These inhibitors are potentially useful for the treatment of hypertension, vascular inflammation, renal inflammation, and lung disease.

### CROSS-REFERENCES TO RELATED APPLICATIONS

This application claims the benefit of U.S. Application No. 60/708,107, filed Aug. 12, 2005, the disclosures of which is incorporated herein by reference.

### STATEMENT AS TO RIGHTS TO INVENTIONS MADE UNDER FEDERALLY SPONSORED RESEARCH OR DEVELOPMENT

This invention was supported in part by grant no. R37 ES02710 awarded by the National Institute of Environmental Health Sciences and grant no. R03 NS050841 awarded by the National Institute of Neurological Disorders and Stroke. The government has certain rights in the invention.

### BACKGROUND OF THE INVENTION

1. Field of the Invention

The present invention generally relates to compounds and methods of inhibiting epoxide hydrolases and treating diseases associated with epoxide hydrolase.

2. Background of the Invention

Hypertension and vascular inflammation are associated with the onset of cardiovascular diseases (CVD), the primary cause of death in our society. Because a large proportion of patients are not responding to current therapies, the next generation of drugs will not only need to reduce blood pressure (BP) but also treat vascular and renal inflammation.

“Epoxide hydrolase” (“EH”) is a ubiquitous enzyme in vertebrates. The EPXH2 gene encodes “soluble epoxide hydrolase” (“sEH”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Unless otherwise specified, as used this background section, the terms “soluble epoxide hydrolase” and “sEH” refer to human sEH.

While highly expressed in the liver, sEH is also expressed in other tissues including vascular endothelium, leukocytes, some smooth muscle and the proximal tubule (Newman et al., Prog. Lipid Res. 44, 1-51 (2005); Draper, A. J., and Hammock, B. D., Toxicol Sci 50, 30-35 (1999); Yu et al., Am. J. Physiol. Renal Physiol. 286, F720-F726 (2004)). This localization reflects the importance of sEH in the metabolism of epoxy fatty acids, such as epoxy-eicosatrienoic acids (EETs) generated by cytochrome P450 epoxygenases (Chacos et al., Arch. Biochem. Biophys. 233, 639-648 (1983)), with critical roles in the regulation of cardiovascular, renal and inflammatory biology (Capdevila, J. H., and Falck, J. R., Biochem. Biophys. Res. Commun. 285, 571-576 (2001); Spector et al., Prog. Lipid. Res. 43, 55-90 (2004); Sun et al., Circ. Res. 90, 1020-1027 (2002); Node et al., Science 285, 1276-1279 (1999)). The hydrolysis of epoxy fatty acids modulates their intracellular fate (Weintraub et al., Am. J. Physiol. 277, H2098-H2108 (1999); Greene et al., Arch. Biochem. Biophys. 376, 420-432 (2000)) and biological activity (Spector et al., Prog. Lipid. Res. 43, 55-90 (2004), Node et al., Science 285, 1276-1279 (1999), Greene et al., Chem. Res. Toxicol. 13, 217-226 (2002); Chen et al., Proc. Natl. Acad. Sci. USA 99, 6029-6034 (2002)). Pharmacological inhibition of sEH has resulted in blood pressure reduction in the spontaneously hypertensive rat (SHR) and in the angiotensin II-induced hypertensive rat model (Yu et al., Circ. Res. 87, 992-998 (2000); Imig et al., Hypertension 39, 690-694 (2002)). In this latter model, sEH inhibition also protects the kidney from hypertension-induced damage (Zhao et al., Am. Soc. Nephrol. 15, 1244-1253 (2004)). Additionally, the deletion of this gene reduces blood pressure in male mice to female levels (Sinal et al., J. Biol. Chem. 275, 40504-40510 (2000)), farther supporting the role of sEH in blood pressure regulation. Thus, Hammock et al. and others have shown that sEH regulates BP and inflammation through EETs hydrolysis. (Tran et al. Biochemistry 44, 12179-87 (2005); Morisseau, C., and Hammock, B. D., Ann. Rev. Pharmacol. Toxicol. 45, 311-333 (2005), Newman et al., Prog. Lipid Res. 44, 1-51 (2005), Argiriadi et al., Proc. Natl. Acad. Sci. USA 96, 10637-10642 (1999); Gomez et al., Biochemistry 43, 4716-4723 (2004)).

At the molecular level, the sEH is a homodimer with a monomeric unit of 62.5 kDa (see FIG. 1; Morisseau, C., and Hammock, B. D., Ann. Rev. Pharmacol. Toxicol. 45, 311-333 (2005)). Analysis of the primary structure suggests that the sEH gene (EPXH2) was produced by the fusion of two primordial dehalogenase genes; the C-terminal sEH domain has high homology to haloalkane dehalogenase, while the N-terminal domain is similar to haloacid dehalogenase (Beetham et al., DNA Cell Biol. 14, 61-71 (1995)). Interestingly both domains possess catalytic activity. The C-terminus of the enzyme has epoxide hydrolase activity (Cterm-EH) which transforms epoxides to their corresponding vicinal diols, specifically eicosatrienoic acids (“EETs”) to dihydroxy derivatives called dihydroxyeicosatrienoic acids (“DHETs”) (Gill et al., Insect Juvenile Hormones: Chemistry and Action (Menn, J. J., and Beroza, M. eds.), pp. 177-189, Academic Press, New York (1972); Morisseau, C., and Hammock B. D., Ann. Rev. Pharmacol. Toxicol. 45, 311-333 (2005).). The N-terminus of the enzyme has phosphatase activity (Nterm-phos).

Recent X-ray crystal structures-of the mouse and human sEH confirmed the gene fusion hypothesis and showed that sEH exhibit a domain-swapped architecture (Argiriadi et al., Proc. Natl. Acad. Sci. USA 96, 10637-10642 (1999); Gomez et al., Biochemistry 43, 4716-4723 (2004)), suggesting a structural role for the N-terminal domain. The C-terminal domain of one subunit interacts with both the C- and N-terminal domain of the other monomer, while the N-terminal domain of one subunit interacts only with the C-terminal of both monomers. Aside from the physical interaction between the two C-terminal domains, no cooperative allosteric effects have been reported for the Cterm-EH activity (Morisseau, C., and Hammock, B. D., Ann. Rev. Pharmacol. Toxicol. 45, 311-333 (2005)). Kinetic analysis revealed a positive cooperative Hill coefficient of ˜2 for the hydrolysis of the monophosphate of dihydroxy stearic acid, suggesting an allosteric interaction between the two monomers (Newman et al., Proc. Natl. Acad. Sci. USA. 100, 1558-1563 (2003)) and suggesting that Nterm-phos activity participates in xenobiotic metabolism (Cronin et al., Proc. Natl. Acad. Sci. USA 100, 1552-1557 (2003)) and/or in the regulation of the physiological functions associated with sEH (Newman et al., Proc. Natl. Acad. Sci. USA. 100, 1558-1563 (2003)). In addition, EH hydrolyzes lipid phosphates which are implicated in the inflammatory response and binding (substrate or inhibitor) to the Nterm-phos reduces the proinflammatory Cterm-EH activity. demonstrating that phosphatase inhibitors are useful to regulate the inflammatory response (Tran et al. Biochemistry 44, 12179-87 (2005);

Common commercial phosphatase inhibitors do not influence Nterm-phos activity (Newman et al., Proc. Natl. Acad. Sci. USA. 100, 1558-1563 (2003)). While sulfates are not substrates for the Nterm-phos activity (Cronin et al., Proc. Natl. Acad. Sci. USA 100, 1552-1557 (2003)), such compounds were recently shown to inhibit two tyrosine phosphatases (Sun et al., J. Biol. Chem. 278, 33392-33399 (2003); Granjeiro et al., Mol. Cell. Biochem. 265, 133-140 (2004)).

Therefore, there is a need to develop potent inhibitors of the Nterm-phos activity. The present invention provides such compounds along with methods for their use and compositions that contain them. The present invention also provides an improved assay for the Nterm-phos activity.

### BRIEF SUMMARY OF THE INVENTION

In one aspect, the present invention provides a method for inhibiting epoxide hydrolase (EH), comprising contacting said soluble epoxide hydrolase with an inhibiting amount of a compound having the structure:

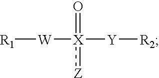

wherein
W is selected from the group consisting of a NH, O, S and CHₙ;
X is selected from the group consisting of As, N, P, Se and S;
Y is selected from the group consisting of NH, O, S and CHₙ;
Z is selected from the group consisting of N, O and S, or Z can be absent;
n is 0, 1, 2 or 3;
R₁ is selected from the group consisting of C₁-C₈alkyl, C₂-C₆ alkenyl, C₂-C₆ alkynyl, heteroC₁-C₈alkyl, C₃-C₁₂cycloalky, aryl and heterocyclyl;
and
R₂ is selected from the group consisting of H, C₁-C₈alkyl, C₂-C₆alkenyl, C₂-C₆alkynyl, heteroC₁-C₈alkyl, C₃-C₁₂cycloalky, aryl and heterocyclyl; wherein each R₁ and R₂ is optionally, independently substituted with from 1 to 6 R₃ substituents selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
the dashed line indicates an optional double bond; and pharmaceutically derivatives thereof.

In another aspect, the present invention provides a method for maintaining the concentration of a biologically active phosphate, said method comprising contacting said soluble epoxide hydrolase with an amount of an inhibitor of the phosphatase activity of said epoxide hydrolase.

In another aspect, the present invention provides a method of increasing sodium excretion in a subject, said method comprising administering to said subject an effective amount of an inhibitor of the phosphatase activity of epoxide hydrolase.

In another aspect, the present invention provides a method of regulating endothelial cell function in a subject, said method comprising administering to said subject an effective amount of an inhibitor of the phosphatase activity of epoxide hydrolase.

In another aspect, the present invention provides a method of treating a disease modulated by soluble epoxide hydrolase, said method comprising administering to the patient a therapeutically effective amount of an inhibitor of the phosphatase activity of epoxide hydrolase.

In another aspect, the present invention provides a compound having the structure:

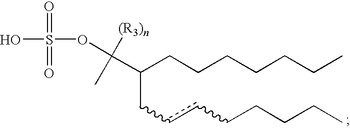

wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

In another aspect, the present invention provides a composition comprising an amount of a compound effective to inhibit or decrease phosphatase activity of sEH.

In another aspect, the present invention provides a use of a compound effective to inhibit or decrease phosphatase activity of sEH effective for the preparation of a medicament for treating a condition in a mammal which is ameliorated by decreasing or inhibiting the phosphatase activity of sEH.

### BRIEF DESCRIPTION OF THE DRAWINGS

FIG. 1: Structure of the human sEH dimer. The N-terminals are in grey and C-terminals in black.

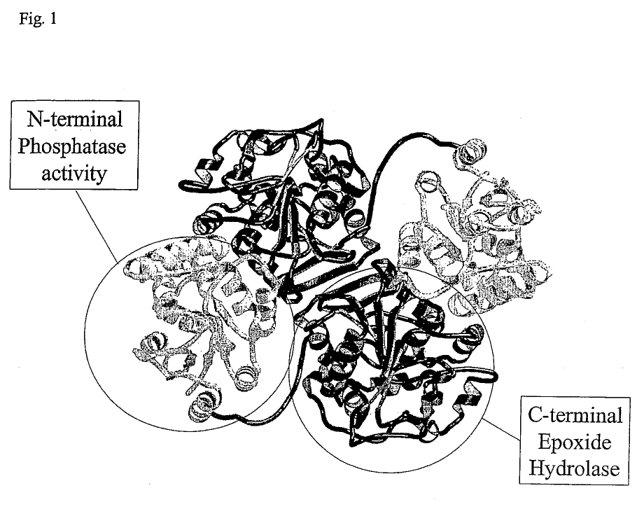

FIG. 2: A: Determination of the dissociation constant of 1 with Human sEH, using Attophos® as substrate. The circles represent the collected data points. The mesh represents the curve resulting from the fitting of the data to equation 1. B: Effect of 1 on Human sEH Nterm-phos activity at a low concentration (1 μM) of Attophos®.

FIG. 3: Determination of the dissociation constant of 1 with Human sEH Cterm-EH activity, using tDPPO as substrate. A: For each inhibitor concentration (0 to 50 μM), the velocity is plotted as a function of the substrate concentration (0 to 30 μM) allowing the determination of an apparent maximal velocity (V_{Mapp}). B: The plotting of 1/V_{Mapp} in function of the concentration of inhibitor permits the determination of K_{I}.

FIG. 4: Hypothetical mechanism of Nterm-phos inhibition by sulfates, sulfonates and phosphonates. The residue numbers are for the human sEH.

### DETAILED DESCRIPTION OF THE INVENTION

### Abbreviations and Definitions:

The abbreviations used herein have their. conventional meaning within the chemical and biological arts.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

The terms “treat”, “treating” and “treatment” refer to any method of alleviating or abrogating a disease or its attendant symptoms.

The term “therapeutically effective amount” refers to that amount of the compound being administered sufficient to prevent or decrease the development of one or more of the symptoms of the disease, condition or disorder being treated.

The term “modulate” refers to the ability of a compound to increase or decrease the function, or activity, of the associated activity (e.g., soluble epoxide hydrolase). “Modulation”, as used herein in its various forms, is meant to include antagonism and partial antagonism of the activity associated with sEH. Inhibitors of sEH are compounds that, e.g., bind to, partially or totally block the enzyme's activity.

The term “compound” as used herein is intended to encompass not only the specified molecular entity but also its pharmaceutically acceptable, pharmacologically active derivatives, including, but not limited to, salts, prodrug conjugates such as esters and amides, metabolites, hydrates, solvates and the like.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, the term “sEH-mediated disease or condition” and the like refers to a disease or condition characterized by less than or greater than normal, sEH activity. A sEH-mediated disease or condition is one in which modulation of sEH results in some effect on the underlying condition or disease (e.g., a sEH inhibitor or antagonist results in some improvement in patient well-being in at least some patients).

“Parenchyma” refers to the tissue characteristic of an organ, as distinguished from associated connective or supporting tissues.

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forceably expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds.), 14th Ed., 1998, McGraw-Hill, New York, pp. 1451-1460 (hereafter, “Harrison's Principles of Internal Medicine”).

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months a year, for two years.

As the names imply, “obstructive pulmonary disease” and “obstructive lung disease” refer to obstructive diseases, as opposed to restrictive diseases. These diseases particularly include COPD, bronchial asthma and small airway disease.

“Small airway disease.” There is a distinct minority of patients whose airflow obstruction is due, solely or predominantly to involvement of the small airways. These are defined as airways less than 2 mm in diameter and correspond to small cartilaginous bronchi, terminal bronchioles and respiratory bronchioles. Small airway disease (SAD) represents luminal obstruction by inflammatory and fibrotic changes that increase airway resistance. The obstruction may be transient or permanent.

The “interstitial lung diseases (ILDs)” are a group of conditions involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with the disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “alkenyl” as used herein refers to an alkyl group as described above which contains one or more sites of unsaturation that is a double bond. Similarly, the term “alkynyl” as used herein refers to an alkyl group as described above which contains one or more sites of unsaturation that is a triple bond.

The term “alkoxy” refers to an alkyl radical as described above which also bears an oxygen substituent which is capable of covalent attachment to another hydrocarbon radical (such as, for example, methoxy, ethoxy, aryloxy and t-butoxy).

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The terms “arylalkyl”, “arylalkenyl” and “aryloxyalkyl” refer to an aryl radical attached directly to an alkyl group, an alkenyl group, or an oxygen which is attached to an alkyl group, respectively. For brevity, aryl as part of a combined term as above, is meant to include heteroaryl as well.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁-C₆ haloalkyl” is mean to include trifluoromethyl, 2,2,2-trifluoroethyl, 4-chlorobutyl, 3-bromopropyl, and the like.

As used herein, the terms “heteroatom” and “hetero” are meant to include oxygen (O), nitrogen (N), Boron (B), phosphorous (P) and sulfur (S). The term “hetero” as used in a “heteroatom-containing alkyl group” (a “heteroalkyl” group) or a “heteroatom-containing aryl group” (a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur or more than one non-carbon atom (e.g., sulfonamide). Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and. U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—; wherein A and B are independently —CH₂—, —O—, —NH—, —S—, —S(O)—, —S(O)₂—, —S(O)₂NR′— or a single bond, and r is an integer of from 1 to 3. One of the-single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically-acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

### Inhibitors of Phosphatase Activity of Epoxide Hydrolase

The present invention also provides novel ligands for the amino terminus active site associated with the phosphatase activity of the enzyme known as soluble epoxide hydrolase. In one embodiment, the compounds are competitive inhibitors which allosterically alter the phosphatase activity. Exemplary classes of these compounds include sulfates, sulfonates, phosphates, pyrophosphates, nitrates, nitrites, and the like and other compounds with the structure set forth below.

In one embodiment, the present invention provides a compound having the structure:

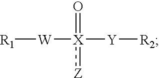

wherein
W is selected from the group consisting of a NH, O, S and CHₙ;
X is selected from the group consisting of As, N, P, Se and S;
Y is selected from the group consisting of NH, O, S and CHₙ;
Z is selected from the group consisting of N, O and S, or Z can be absent;
n is 0, 1, 2 or 3;
R₁ is selected from the group consisting of C₁-C₈alkyl, C₂-C₆ alkenyl, C₂-C₆ alkynyl, heteroC₁-C₈alkyl, C₃-C₁₂cycloalky, aryl and heterocyclyl;
and
R₂ is selected from the group consisting of H, C₁-C₈alkyl, C₂-C₆alkenyl, C₂-C₆alkynyl, heteroC₁-C₈alkyl, C₃-C₁₂cycloalky, aryl and heterocyclyl; wherein each R₁ and R₂ is optionally, independently substituted with from 1 to 6 R₃ substituents selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl; the dashed line indicates an optional double bond; and pharmaceutically derivatives thereof.

In one embodiment, W is NH. In another embodiment, W is O. In another embodiment, W is S. In another embodiment, W is CHₙ. In another embodiment, W is NH. In another embodiment, W is O. In another embodiment, W is S. In another embodiment, W is CHₙ. In another embodiment, Y is NH. In another embodiment, Y is O. In another embodiment, Y is S. In another embodiment, Y is CHₙ. In another embodiment, Z is N. In another embodiment, Z is O. In another embodiment, Z is S. In another embodiment, Z is absent. In another embodiment, W, Y and Z is O; and X is S. In another embodiment, n is 1. In another embodiment, n is 2. In another embodiment, n is 3. In another embodiment, R¹ is alkyl. In another embodiment, R¹ is cycloalkyl. In another embodiment, R¹ is aryl. In another embodiment, R¹ is heterocyclyl. In another embodiment, R² is alkyl. In another embodiment, R² is cycloalkyl. In another embodiment, R² is aryl. In another embodiment, R² is heterocyclyl. In another embodiment, R¹ is alkyl. In-another embodiment, R² is hydrogen. In another embodiment, W, Y and Z is O; X is S; R¹ is alkyl; and R² is hydrogen.

In another embodiment, the inhibitor has the structure:

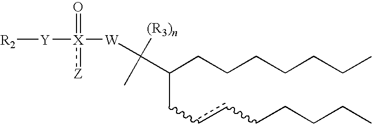

wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

In another embodiment, the inhibitor has the structure:

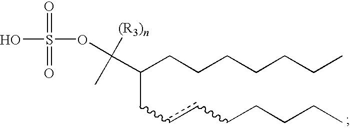

wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

In one embodiment, n R3 is selected from the group consisting of C₁-C₈alkyl, hydroxyl, carboxy and C₁-C₈alkylcarboxy.

In one embodiment, a compound is selected from the group consisting of:

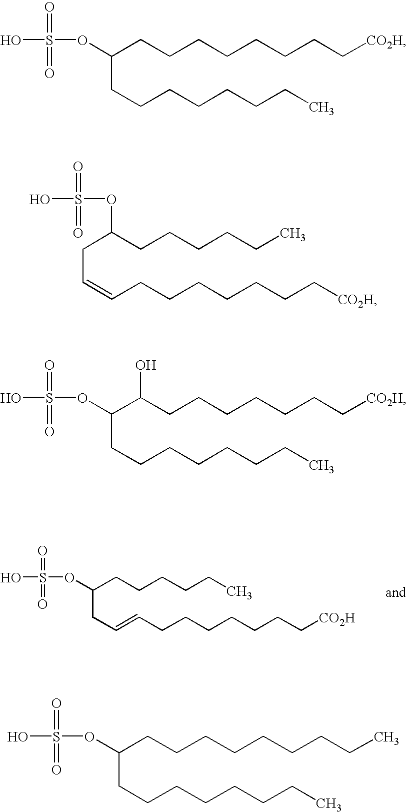

and pharmaceutically acceptable derivatives thereof.

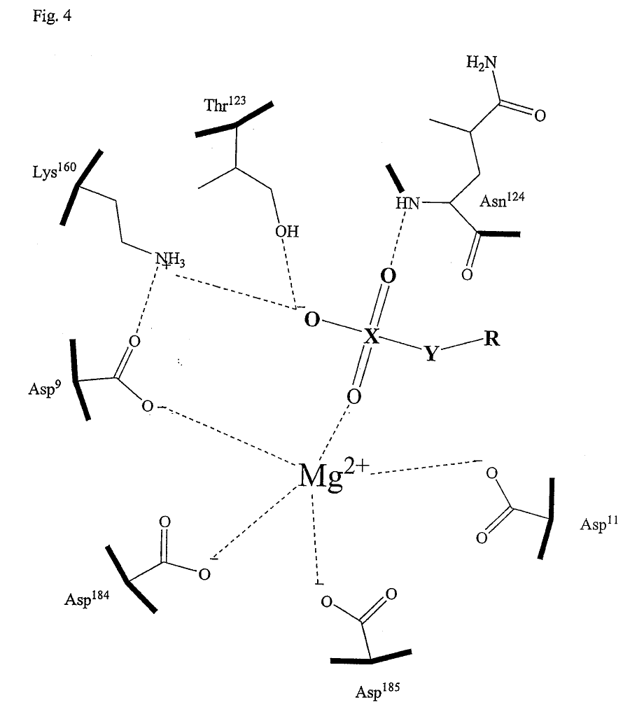

The X-ray crystal structure of the human sEH shows that the conserved catalytic residues within the N-terminal domain, includes D9X10D11X12V13, as well as T123, K160, D184, and D185. Furthermore, these residues were found to be properly oriented for potential catalytic activity (see Gomez et al. (2004), Biochemistry 43, 4716-4723; Cronin et al., (2003) Proc. Natl. Acad. Sci. USA 100, 1552-1557). Crystal structure of the human sEH, and mutation of the putative catalytic aspartate (D9) that abolished Nterm-phos activity, suggests that Nterm-phos follows the general mechanism of the HAD superfamily phosphatases, which involves the formation of a covalent phosphoenzyme intermediate with Asp9 (FIG. 4). Furthermore, the crystal structure points out two special features of Nterm-phos. First, as shown on FIG. 4, the catalytic cavity contains the polar residue Arg99, which is closed to Asp11. Second, this Arg99 is at the beginning of a ˜14 Å long hydrophobic tunnel sufficiently large to accommodate the binding of Nterm-phos substrates, and whose other end is near the interface of the N-and C-terminal domains. Because particularly substituted sulfates, sulfonates and phosphonates inhibit the phosphatase activity of sEH, the present invention also provides inhibitors that mimic-the binding of the phosphate substrate in the catalytic cavity (see FIG. 4). Thus in another embodiment, the present invention provides methods wherein the inhibitor is complementary to a portion of the phosphatase active site of epoxide hydrolase

Further, in addition to the above compounds, prodrug derivatives can be designed for practicing the invention (Gilman et al., The Pharmacological Basis of Therapeutics, 7^{th} Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the alcohols and acids described here or the single active hydrogen present on the W or Y nitrogen is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al., Journal of Agricultural and Food Chemistry, 26(3) :550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme substrate so that there is a stable interaction with the enzyme catalytic site. The inhibitors appear to form hydrogen bonds with the cofactor and amino acids of the catalytic site.

Means for preparing such compounds are generally shown in Scheme I below.

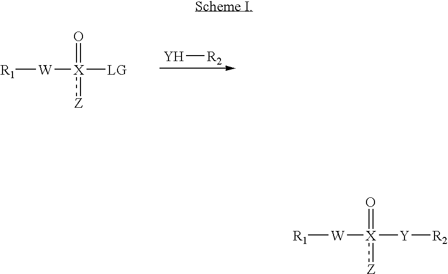

wherein LG represents a leaving group such as a halogen. As shown on Scheme 1, the synthesis of the inhibitors can be done in a simple procedure following the steps used to synthesize Nterm-phos substrates (see Newman et. al., Proc. Natl. Acad. Sci. USA. 100, 1558-1563 (2003); Tran et al. Biochemistry 44:12179-12187 (2005)). The activated mineral acid can be added to the appropriate alcohol to yield a mineral ester. The replacement of the alcohol by an amine or a thiol will lead to the formation of amides and thioesters, respectively. Because all mineral acids are not available in an activated form, the activated acids can be generated in situ through reaction with trichloroacetonitrile in basic conditions. The mixture can be purified by flash chromatography on silica gel or a C18-reverse phase column. The structure of the purified compound can be confirmed by NMR and mass spectrometry.

### Assays for Phosphatase Activity of Epoxide Hydrolase

In one embodiment, the inhibiting is inhibiting the phosphatase activity of said epoxide hydrolase. Thus, the invention also provide methods for assaying for phosphatase activity of epoxide as a diagnostic assay to identify individuals at increased risk for hypertension and/or those that would benefit from the therapeutic methods of the invention. A suitable assays are described below. The enzyme also can be detected based on the binding of specific ligands to the catalytic site which either immobilize the enzyme or label it with a probe such as luciferase, green fluorescent protein or other reagent.

The assays of the invention are carried out .using an appropriate sample from the patient. Typically, such a sample is a blood sample.

Where the modified activity of the complexed epoxide hydrolase is enzyme inhibition, then particularly preferred compound embodiments have an IC₅₀ (inhibition potency or, by definition, the concentration of inhibitor which reduces enzyme activity-by 50%) of less than about 500 M.

### Methods of Treating Diseases Modulated by Soluble Epoxide Hydrolases and Therapeutic Administration:

The methods and compounds of the present invention are useful in treating diseases mediated by EH while simultaneously increasing sodium excretion, reducing vascular and renal inflammation, and reducing male erectile dysfunction As shown below (see Examples and Figures), Nterm-Phos hydrolyzed several natural lipid phosphates implicated in numerous cellular responses including cellular proliferation, cell migration, platelet aggregation, and arteriosclerosis and therefore important in the regulating the inflammatory process. Since these compounds are anti-hypertensive and anti-inflammatory, altering their concentration can lead to reduced blood pressure and reduced vascular and renal inflammation.

Thus in another aspect, the present invention provides methods of treating diseases, especially those modulated by soluble epoxide hydrolases (sEH). The methods generally involve administering to a subject in need of such treatment an effective amount of a compound, above. The dose, frequency and timing of such administering will depend in large part on the selected therapeutic agent, the nature of the condition being treated, the condition of the subject including age, weight and presence of other conditions or disorders, the formulation being administered and the discretion of the attending physician. In one embodiment, the compositions and compounds of the invention and the pharmaceutically acceptable salts thereof are administered via oral, parenteral, subcutaneous, intramuscular, intravenous or topical routes. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and either a compound of the invention or a pharmaceutically acceptable salt of the compound.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the compounds of the invention is employed in treatment. The dosage of the specific compound for treatment depends on many factors that are well known to those skilled in the art. They include for example, the route of administration and the potency of the particular compound. An exemplary dose is from about 0.001 mg/kg to about 100 mg/kg body weight of the mammal. Generally, the compounds are administered in dosages ranging from about 2 mg up to about 2,000 mg per day, although variations will necessarily occur depending, as noted above, on the disease target, the patient, and the route of administration. Dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day. An exemplary dose is from about 0.001 mg/kg to about 100 mg/kg body weight of the mammal. The dosage employed for the topical administration will, of course, depend on the size of the area being treated.

It has previously been shown that inhibitors of soluble epoxide hydrolase (“sEH”) can be used to treat a number of conditions and diseases. See, e.g. references supra and U.S. Pat. No. 6,351,506. Thus in one embodiment, the present invention provides a method for maintaining the concentration of a biologically active phosphate, said method comprising contacting said soluble epoxide hydrolase with an amount of an inhibitor of the phosphatase activity of said epoxide hydrolase.

In another embodiment, the present invention provides a method of increasing sodium excretion in a subject, said method comprising administering to said subject an effective amount of an inhibitor of the phosphatase activity of epoxide hydrolase.

In another embodiment, the present invention provides a method of regulating endothelial cell function in a subject, said method comprising administering to said subject an effective amount of an inhibitor of the phosphatase activity of epoxide hydrolase.

In another embodiment, the present invention provides a method of treating a disease modulated by soluble epoxide hydrolase, said method comprising administering to the patient a therapeutically effective amount of an inhibitor of the phosphatase activity of epoxide hydrolase. In one embodiment, the disease is selected from the group consisting of hypertension, inflammation, adult respiratory distress syndrome; diabetes or its complications; end stage renal disease; Raynaud syndrome, arthritis, erectile dysfunction, renal deterioration, nephropathy, high blood pressure, obstructive pulmonary disease, interstitial lung disease and asthma. In one embodiment, the disease is inflammation. In one embodiment, the inflammation is selected from the group consisting of renal inflammation, vascular inflammation, lung inflammation, endothelial cell inflammation.

### Combination Therapy

As noted above, the compounds of the present invention will, in some instances, be used in combination with other therapeutic agents to bring about a desired effect. Selection of additional agents will, in large part, depend on the desired target therapy (see, e.g. Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound having the general structure of formula 1 and one or more additional active agents, as well as administration of a compound of formula 1 and each active agent in its own separate pharmaceutical dosage formulation. For example, a compound of formula 1 and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, a compound of formula 1 and one or more additional active agents can be administered at essentially the same time (i.e., concurrently), or at separately staggered times (i.e., sequentially). Combination therapy is understood to include all these regimens.

Without further elaboration, it is believed that one skilled in the art can, using the preceding description, practice the present invention to its fullest extent. The following detailed examples describe how to prepare the various compounds and/or perform the various processes of the invention and are to be construed. as merely illustrative, and not limitations of the preceding disclosure in any way whatsoever. Those skilled in the art will promptly recognize appropriate variations from the procedures both as to reactants and as to reaction conditions and techniques.

### EXAMPLES

### Abbreviations

sEH: soluble epoxide hydrolase; Cterm-EH: C-terminal epoxide hydrolase; Nterm-Phos: N-terminal phosphatase.

### General Materials and Methods

Fatty acids were purchased from NuChek Prep (Elysian, Minn.). HPLC grade chloroform (CHCl₃), triethylamine (TEA) and glacial acetic acid were purchased from Fisher Scientific (Pittsburgh, Pa.). OmniSolv™ acetonitrile (ACN) and methanol (MeOH) were purchased from EM Science (Gibbstown, N.J.). Compounds 1 to 5 were synthesized through the in situ generation of an activated sulfoimidate which was used to sulforylate hydroxy fatty acids following a method similar to the one used previously to synthesize lipid phosphates (Newman et al., Proc. Natl. Acad. Sci. USA. 100, 1558-1563 (2003); Ullman, B., and Perlman, R. L., Biochem. Biophys. Res. Commun. 63, 424-430 (1975)). As an example, synthesis of compound 1 is described below. In addition, reaction yield and high resolution mass spectrometry data for compound 1 to 5 are given in Table 1. ¹H-NMRs were performed using a Mercury 300 NMR (Varian; Walnut Creek, Calif.). High resolution mass spectra were acquired on a time-of-flight mass spectrometer (Micromass LCT; Manchester, UK) using negative mode electrospray ionization (ESI) and leucine enkephalin as a lock mass compound. Chemical purity was estimated at >95% for each compound based on ¹H-NMR spectra and ESI-LC/MS analyses. Negative mode electrospray ionization showed a single peak, while positive mode confirmed TEA as the only ESI-LC/MS detectable secondary component. Compound 6 was synthesized previously in the laboratory (Newman et al., Proc. Natl. Acad. Sci. USA. 100, 1558-1563 (2003)). Compounds 7 to 37 were purchased from either Sigma (St. Louis, Mo.) or Aldrich Chemical Co. (Milwaukee, Wis.), except for compound 9 which was provided by Promega (Madison, Wis.), compound 10 which was provided by City Chemicals (West-Haven, Conn.), and compound 33 that was purchased from Polycarbon Industries, Inc. (Devens, Mass.).

TABLE 1
Hydroxy lipid sulfate characterization

| | | | Yield | Mass |
|---|---|---|---|---|
| Name | Structure | No. | (%) | ([M − H]⁻) |
| 10-Sulfonooxy- octadecanoic acid | 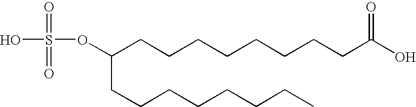 | 1 | 25 | 379.2165 (379.2233) |
| 9/10- Sulfonooxy- hydroxy- octadecanoic acid | 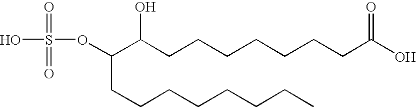 | 2 | 16 | 395.2104 (395.2182) |
| 9-Octadecanyl- sulfate | 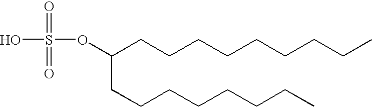 | 3 | 2 | 349.2384 (349.2491) |
| 12-Sulfonoxy- cis-9- octadecenoic acid | 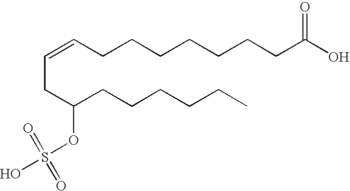 | 4 | 7 | 377.2051 (377.2076) |
| 12-Sulfonoxy- trans-9- octadecenoic acid | 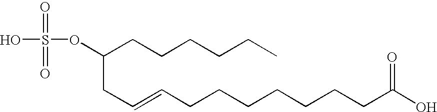 | 5 | 1 | 377.1929 (377.2076) |

Analyte purity was >95%. While a single structure is shown, diol sulfate 2 is a 1:1 mixture of the monosulfate of each possible alcohol. Measured anionic masses are shown with theoretical masses in parentheses.

### Enzyme Preparations.

Recombinant human sEH (HsEH) was produced in a baculovirus expression system (Beetham et al., Arch. Biochem. Biophys. 305, 197-201 (1993)) and purified by affinity chromatography (Wixtrom et al., Anal. Biochem. 169, 71-80 (1983)). The preparations were at least 97% pure as judged by sodium dodecyl sulfate-polyacrylamide gel electrophoresis and scanning densitometry. No detectable esterase or glutathione transferase activities were observed. Recombinant mouse-eared cress sEH was produced in a baculovirus expression system and purified as described (Morisseau et al., Arch. Biochem. Biophys. 378, 321-332 (2000)). The nucleotide sequence of the C-terminal region (Mel²³⁷-Met⁵⁵⁴) of human sEH was amplified by PCR using 5′-CCGGAATTCATGAGCCATGGGTACGTGA-3′ as forward primer and 5′-ACGCGTCGACCTACATCTTTGAGACCACCG-3′ as reverse primer. The resulting band was gel purified and restricted with EcoR1 and Sal1, which were introduced by the primers, and the restricted fragment sub-cloned into the multiple cloning site of the pFastBac1 vector (Invitrogen). After verification of the obtained nucleotide sequence, the recombinant pFastBac1 plasmid was introduced in competent DH10bac cells leading to the formation of a recombinant plasmid containing the DNA insert. The isolated recombinant plasmid was used to produce recombinant baculovirus in Sf21 cells following procedures recommended by the manufacturer. The truncated HsEH was produced in high-5 Trichloplusia ni cell cultures following published procedures (Beetham et al., Arch. Biochem. Biophys. 305, 197-201 (1993)). Seventy-two hours post infection, the cells were collected by centrifugation (2,000 g×20 min). The cell pellet was then suspended in a sodium phosphate buffer (76 mM, pH 7.4) containing 1 mM of phenylmethylsulfonyl fluoride, EDTA and dithiothretol. The suspension was then homogenized by Polytron at 9,000 rpm for 1 min and centrifuged (10,000 g×20 min). The resulting supernatant was frozen at −80° C. until used for experiments. The human placental alkaline phosphatase (AP_{HP}) was obtained from Sigma. Protein concentrations were quantified with the Pierce BCA assay (Pierce; Rockford, Ill.) using Fraction V bovine serum albumin (BSA) as the calibrating standard.

### Example 1

### Enzymatic Assays.

Nterm-Phos activity was measured in Bis-Tris HCl buffer (25 mM PH 7.0) containing 0.1 mg/mL of Fraction V BSA and 1 mM of MgCl₂ (buffer A) at 30° C. For compounds 8 and 9, the appearance of the fluorescent products was followed kinetically for 5-10 minutes on Spectromax M2 (Molecular Devices, Sunnyvale, Calif.) at the emission and excitation wavelengths recommended by the manufacturers. AP_{HP} activity was measured as described using 7 as substrate (Newman et al., Proc. Natl. Acad. Sci. USA. 100, 1558-1563 (2003)). Compounds 34-37 were incubated separately with the enzyme for a given time, and then the reaction was stopped by the addition of 100 μL of methanol. The reaction products for compounds 34-37 were extracted with 500 μL of ethyl acetate. The quantification of the alcohol products was performed by LC/MS analysis of 2 μL of the organic phase. The Cterm-EH activity was measured as described previously in buffer A, using either racemic 4-nitrophenyl-trans-2,3-epoxy-3-phenylpropyl carbonate (Dietze et al., Anal. Biochem. 216, 176-187 (1994)) or racemic [³H]-trans-1,3-diphenylpropene oxide as substrate (Borhan et al., Anal. Biochem. 231, 188-200 (1995)). This latter substrate was also used to measure the activity of the cress sEH and the truncated human sEH.

### Example 2

Nterm-phos hydrolysis of poly-isoprenyl pyrophosphates (PIPPs).

This example shows that Nterm-phos hydrolyzes poly-isoprenyl pyrophosphates (PIPPs). Table 2 shows that the Nterm-phos hydrolyzes poly-isoprenyl pyrophosphates (PIPPs), such as farnesyl pyrophosphate (FPP), presqualene diphosphate (PSDP) and presqualene monophosphate (PSMP), and lysophosphatidic acids (LPAs), such as 1-oleyl-2-hydroxyglycerol-3-phosphate (OGP). PIPPs are natural anti-inflammatory lipid signals that influence the progress and resolution of vascular inflammation, while LPAs have been implicated in numerous cellular responses including cellular proliferation, cell migration, platelet aggregation, and arteriosclerosis, a leading cause of cardiovascular disease (CVD). Put together, these results a role of Nterm-phos in the inflammation response that could be complementary of the Cterm-EH for the possible treatment of CVD.

TABLE 2
Specific activity of Human Nterm-phos for various lipid-phosphates.

| | Spec. Act. |
|---|---|
| | (nmol · min⁻¹ · |
| Name | mg⁻¹) |
| Farnesyl-pyrophosphate (FPP) | 32 ± 7 |
| Pre-squalene diphosphate (PSDP) | 30 ± 15 |
| Pre-squalene monophosphate (PSMP) | 54 ± 22 |
| Sphingosine-1-phosphate (S1P) | 1.1 ± 0.1 |
| N-Octyl-ceramide-1-phosphate (OCP) | <0.3 |
| 1-Oleyl-2-hydroxyglycerol-3-phosphate (OGP) | 67 ± 3 |
| 1,2-Dioleoyl-glycerol-3-phosphate (DOGP) | <0.3 |

Results are average ± SD of three separated experiments. Activities were determined by quantifying the amount of phosphoric acid form after incubation with the enzyme at 30° C.

As shown in Table 3, the potential natural substrates for Nterm-phos, especially some PIPP and LPA, have an inhibitory effect on the Cterm-EH activity. It suggests that through an allosteric mechanism Nterm-phos regulates the Cterm-EH activity. The recently developed potent chemical inhibitors for Nterm-phos are allosteric competitive inhibitors with a K_{I} in the hundred nanomolar range. Put together, it suggests that these chemical inhibitors of Nterm-phos be used to reduce inflammation by 1) reducing the hydrolysis of anti-inflammatory lipid phosphates (PIPP and LPA) by Nterm-phos activity, and 2) by inhibiting the proinflammatory Cterm-EH activity of the human sEH.

TABLE 3
Inhibition of Human Cterm-EH activity by various lipid-phosphates (LP).

| | [LP] | Inhibition |
|---|---|---|
| Name | (μM) | (%) |
| Geranyl-geranyl-pyrophosphate | 10 | 45 |
| Farnesyl-pyrophosphate (FPP) | 10 | 60 |
| Pre-squalene diphosphate (PSDP) | 14 | 86 |
| Pre-squalene monophosphate (PSMP) | 14 | 30 |
| Sphingosine-1-phosphate (S1P) | 40 | 2 |
| N-Octyl-ceramide-1-phosphate (OCP) | 50 | 31 |
| 1-Oleyl-2-hydroxyglycerol-3-phosphate (OGP) | 50 | 62 |

Cterm-EH activity was measured using a fluorescent assay (Jones et al., Anal. Biochem. 343, 66-75 (2005)).

### Example 3

10-Sulfonooxy-octadecanoic acid 1.

In a small reaction vial, 100 mg of 10-hydroxy-octanoic acid was dissolved in 0.8 mL of acetonitrile and enriched with 150 μL of triethylamine, followed by 60 μL of trichloroacetonitrile and 40 μL of 100% sulfuric acid. The mixture was stirred at 50° C. for 2 hours. The acetonitrile was then evaporated, and the resulting residue was dissolved in 10 mL of 1:4 methanol/water (v/v). The mixture was purified using a 1 g C18 solid phase extraction cartridge (SPE; Varian, Walnut Creek, Calif.) equilibrated with water. The sulfurylated product was eluted from the column with 2:3 methanol/water (v/v). Fractions were screened for purity by ESI-LC/MS and solvent removed under vacuum to yield 32 mg (25% yield) of a yellow brown waxy solid. Analysis revealed that the target compound was obtained as a triethylamine (TEA) salt. ¹H-NMR(CDCl3:CD3OD 1:1): δ 4.35 (m, 1H, C10), 3.18 (dd, J=7.5 Hz, 6H, CH2s of TEA), 2.29 (t, J=7.2 Hz, 2H, C2), 1.63 (m, 11H, CH3s on TEA and C3), 1.30 (n, 26H, C4-C9 and C11-C17) and 0.88 (t, J=6.9 Hz, 3H, C18) ppm. High-resolution MS m/z: 379.2165 (Th. 379.2233).

### Example 4

### Dephosphorylation Quantification.

The quantification of geraniol, farnesol, and geranylgeraniol, the products from dephosphorylation of compounds 34-37, was performed using HPLC with ESI and tandem mass spectrometric detection (MS/MS). The Shimadzu ASP10 HPLC system (Shimadzu Scientific Instruments, Columbia, Md.) was set at a flow rate of 0.2 mL/min, and a 2.1×30 mm XTerra™ MS C₁₈ 3.5 μm column (Waters, Milford, Mass.) held at 20° C. The samples were kept at 10° C. in the auto-sampler. The injection volume was 2 μL. A solvent system consisting of water with 0.1% formic acid (solvent A) and acetonitrile with 0.1% formic acid (solvent B) was used and set at a flow rate of 0.25 mL/min. The analytes were separated using a gradient program starting with a solvent composition of 40% Solvent B ramped using a linear gradient for 7 min to 100% Solvent B, held for 0.5 min. Compound 37 was analyzed by direct injections of 3 μL sample into the mass spectrometer at 0.25 mL/min flow rate of 10% A and 90% B. Pyrophosphate was analyzed by direct injection of 5 μL sample into the mass spectrometer at 0.05 mL/min flow rate of 50% A and 50% B.

Analytes were detected by electrospray ionization—tandem quadrupole mass spectrometry in multiple reaction monitoring mode (MRM) using a Quattro Premier tandem quadrupole mass spectrometer (Micromass, Manchester, UK). Nitrogen gas flow rates were fixed with a cone gas flow of 25 L/h and a desolvation gas flow of 700 L/h. Electrospray ionization of geraniol, farnesol and geranylgeraniol was performed in positive mode with a capillary voltage fixed at 3.20 kV and a cone voltage fixed at 25 V using a source temperature of 125° C. and a desolvation temperature of 350° C. Capillary voltage and cone voltage were optimized in an infusion experiment. Intensities of analyte molecular ions [M+H]+ were low at 10 μM concentration of infused standards. However, intense [M+H−18]⁺ ions were produced in the source due to water loss. Therefore, these ions were selected as precursor ions to set MRM acquisition mode. Monitored transitions were 137>95 m/z for geraniol, 205>121 m/z for farnesol, and 273>149 m/z for geranylgeraniol at collision voltage of 15 V for all analytes. Argon was used as collision gas (2.2×10⁻³ Torr). Electrospray ionization of compound 37 and pyrophosphate was performed in negative ionization mode at the same instrument conditions described above using MRM transition 301>97 m/z and 177>79 m/z respectively.

Concentrations of geraniol, farnesol, geranylgeraniol, and compound 37 were quantified using external standard calibration. Calibration curves for geraniol, farnesol and geranylgeraniol contained six points from 0.03 to 10.0 μM and were linear (r²>0.99). Calibration curve for compound 37 contained seven points from 0.15 to 15 μM and had a good linear fit (²=0.97). Chromatogram integration and analyte quantification was performed with QuantLynx module of the MassLynx 4.0 software (Micromass, Manchester, UK). Limit of detection for pyrophosphate was found injecting serial dilutions of the standard and was estimated to be 0.01 μM at 5 μL sample volume.

### Example 5

### Inhibition Experiments.

IC₅₀s for the Nterm-phos activity were determined using Attophos®, 9, as substrate. Human sEH (400 nM) was incubated with inhibitors for 5 min in buffer A at 30° C. prior to substrate addition ([S]=50 μM). IC₅₀s for the Cterm-EH activity were determined using racemic 4-nitrophenyl-trans-2,3-epoxy-3-phenylpropyl carbonate as substrate as described (Dietze et al., Anal. Biochem. 216, 176-187 (1994); Morisseau et al., Arch. Biochem. Biophys. 356, 214-228 (1998)). Human sEH (100 nM) was incubated with inhibitors for 5 min in buffer A at 30° C. prior to substrate addition ([S]=50 μM). By definition, IC₅₀ is the concentration of inhibitor that reduces enzyme activity by 50%. The IC₅₀ was determined by regression of at least five datum points with a minimum of two points in the linear region of the curve on either side of the IC₅₀ value.

For the Nterm-phos activity, dissociation constants were determined using Attophos® as substrate. Compound 1 at concentrations between 0 and 25 μM was incubated in triplicate for 5 min in at 30° C. with 200 μl of purified human sEH at 40 nM in buffer A. Substrate (3.1<[S]_{final}<100 μM) was then added. Velocity was measured as described above. The results were fitted to the equation (Eq. 1) corresponding to a competitive allosteric inhibition of two catalytic sites (Segel, I. H., Enzyme kinetics: behavior and analysis of rapid equilibrium and steady state enzyme systems, Wiley, New York (1993)), allowing the simultaneous determination of K_{I}, α and K_{S}. Resolution of the non-linear equation was performed using Sigma Plot (SPSS Science; Chicago, Ill.).

$$v = \frac{{V}_{M} ( [ S ] + \frac{{[ S ]}^{2}}{α \cdot {K}_{S}} + \frac{[ S ] \cdot [ I ]}{α \cdot {K}_{I}} )}{( \begin{matrix}{K}_{S} + 2 \cdot [ S ] + \frac{{[ S ]}^{2}}{α \cdot {K}_{S}} + \frac{2 \cdot [ S ] \cdot [ I ]}{α \cdot {K}_{I}} + \\ \frac{2 \cdot {K}_{S} \cdot [ I ]}{{K}_{I}} + \frac{{K}_{S} \cdot {[ I ]}^{2}}{α \cdot {K}_{I}^{2}}\end{matrix} )} \qquad Eq . 1$$

For the Cterm-EH activity, dissociation constants were determined using racemic [³H]-trans-1,3-diphenylpropene oxide as substrate. Compound 1 at concentrations between 0 and 50 μM was incubated in triplicate for 5 min at 30° C. with 100 μl of purified human sEH at 1 nM in buffer A. Substrate (2.5<[S]_{final}<30 μM) was then added. Velocity was measured as described (Borhan et al., Anal. Biochem. 231, 188-200 (1995)). For each inhibitor concentration, the plots of the velocity as a function of the substrate concentration allowed the determination of apparent kinetic constants (K_{Mapp} and V_{Mapp}) (Segel, I. H., Enzyme kinetics: behavior and analysis of rapid equilibrium and steady state enzyme systems, Wiley, New York (1993)). Resolution of the non-linear Michaelis equation was performed using Sigma Plot (SPSS Science; Chicago, Ill.). The plot of 1/V_{Mapp} as a function of the inhibitor concentration allows the determination of K_{I} when I/V_{Mapp}=0. Results are mean±standard deviation of three separate determination of K_{I}.

### Nterm-Phos Assay Optimization.

Phosphate esters of dihydroxy-fatty acids such as compound 6 (see Table 4), are good substrates for Nterm-phos, however they are difficult to synthesize (reaction yield ˜1%) and detection of the hydrolysis products require chromatographic separation and mass spectral detection (Newman et al., Proc. Natl. Acad. Sci. USA. 100, 1558-1563 (2003)). On the other hand, the readily available p-nitrophenyl phosphate, 7, is a relatively poor substrate for the targeted activity with a low V_{M} to Kₘ ratio (Table 2). Therefore, in order to obtain a more facile assay to test for Nterm-Phos activity, we tested two fluorescent phosphatase substrates. The 4-methyl-umbeliferol phosphate, 8, is a poor substrate for the human sEH, as it was for the rat sEH (Cronin et al., Proc. Natl. Acad. Sci. USA 100, 1552-1557 (2003)). On the other hand, Attophos®, 9, is a good substrate for the Nterm-phos, with a Kₘ value 5-fold lower than that for compound 6, the best substrate previously reported (Newman et al., Proc. Natl. Acad. Sci. USA. 100, 1558-1563 (2003)). While 9 is hydrolyzed 50-fold slower than 6, the high sensitivity of the fluorescent reporter allows the use of 5-fold less enzyme (40 nM instead of 180 nM). Furthermore, we were able to execute the fluorescent assay in a 96-well format, permitting the rapid screening of chemicals for Nterm-phos inhibition.

TABLE 4
Catalytic activity of human sEH for several phosphate substrates.

| | | | V_{M} | | V_{M}/Kₘ |
|---|---|---|---|---|---|
| | | Kₘ | (nmol · min⁻¹ · | Hill | (nmol · min⁻¹· |
| Name | No. | (μM) | mg⁻¹) | coefficient | mg⁻¹ · μM⁻¹) |
| threo-9/10-phosphonooxy- | 6 | 20.9^{a} | 338^{a} | 1.9^{a} | 16.1 |
| octadecanoic acid | | | | | |
| p-Nitrophenyl phosphate | 7 | 1,600^{a} | 57.6^{a} | 1.0^{a} | 0.04 |
| 4-Methyl-umbeliferyl | 8 | 210 ± 30 | 7.9 ± 1.7 | 1.1 ± 0.1 | 0.04 ± 0.01 |
| phosphate | | | | | |
| AttoPhos ® | 9 | 3.6 ± 0.8 | 7.0 ± 0.1 | 1.6 ± 0.1 | 1.9 ± 0.2 |

^{a}data from (Newman et al., Proc. Natl. Acad. Sci. USA. 100, 1558-1563 (2003)).

### Nterm-Phos Inhibition.

Phosphoesters of hydroxyl-fatty acids, such as 6, are good substrates for the Nterm-phos activity (Newman et al., Proc. Natl. Acad. Sci. USA. 100, 1558-1563 (2003)). Moreover, sulfates acting as inhibitors of phosphatases have also been reported (Sun et al., J. Biol. Chem. 278, 33392-33399 (2003); Granjeiro et al., Mol. Cell. Biochem. 265, 133-140 (2004); Scott et al., Pharm. 41, 1529-1532 (1991)). Therefore, we hypothesize that replacing the phosphate moiety by a sulfate would yield potent inhibitors for Nterm-phos activity. Following a procedure similar to that used to make phosphoesters (Newman et al., Proc. Natl. Acad. Sci. USA. 100, 1558-1563 (2003)), we synthesized five sulfate derivatives of hydroxyl-fatty acids (Table 1). Using Attophos® as a reporting substrate, we measured the effects of these compounds as well as a series of commercials sulfates and sulfonates, on the Nterm-phos activity. As hypothesized, lipid sulfates are effective inhibitors of Nterm-phos (data shown in Table 5). Interestingly, the structure activity obtained with the sulfate inhibitors differs from what was observed with the corresponding phosphate substrates (Newman et al., Proc. Natl. Acad. Sci. USA. 100, 1558-1563 (2003)). Compared to compound 1, the presence of the hydroxyl group alpha to the sulfate in compound 2 does not increase the potency while a corresponding alpha hydroxy improved the substrate affinity of lipid phosphates (Newman et al., Proc. Natl. Acad. Sci. USA. 100, 1558-1563 (2003)). The removal of the acid function in compound 3 did not affect the inhibition potency. Furthermore, the sulfate of trans-ricinelaidate, 5, gives a 10-fold higher inhibition than the cis-isomer ricinoleate, 4, and the phosphate of the cis-isomer is hydrolyzed 6-fold faster than the trans-isomer (Newman et al., Proc. Natl. Acad. Sci. USA. 100, 1558-1563 (2003)). In comparison to compound 1, removal of the sulfate group from the middle of the alkyl chain and placing it on the carbon next to the acid function, as in compound 10, resulted in a two-fold loss of inhibition potency. A terminal sulfate function with a shorter alkyl chain, 11, resulted in a potent inhibitor, demonstrating the importance of the presence of a hydrophobic group to inhibitor potency and that the acid function is not necessary. Compared to 11, the replacement of the sulfate group by a sulfonate, as in compound 12, results in an inhibitor with similar potency, suggesting that sulfonates and sulfates are both potent inhibitors of Nterm-phos. Compared to compound 11, the replacement of the alkyl chain by an aromatic group, as in compound 13, resulted in a total loss of potency. Interestingly, compound 13 was found not to be a substrate for the rat sEH, and the corresponding phosphate, compound 7, is a poor substrate (Cronin et al., Proc. Natl. Acad. Sci. USA 100, 1552-1557 (2003); Newman et al., Proc. Natl. Acad. Sci. USA. 100, 1558-1563 (2003)). Good inhibition was obtained for compound 14 which has a sulfate group on position 3 of the A ring and a sulfonate on the alkyl tail of the sterol structure. The fact that taurocholic acid, compound 15, which has only the sulfonate function, suggests that the observed inhibition by compound 14 is due to the presence of the sulfate group on the A ring. Compared to compound 11, the replacement of the alkyl chain by hydrophilic groups, such as compounds 16-19, resulted in a total loss of inhibition potency.

TABLE 5
Effect of sulfates on Human sEH Nterm-Phos activity.

| Name | No. | IC₅₀ (μM)^{a} |
|---|---|---|
| 10-Sulfonooxy-octadecanoic acid | 1 | 5.9 ± 1.6 |
| 9/10-Hydroxy-sulfonooxy- | 2 | 17.5 ± 1.6 |
| octadecanoic acid | | |
| 9-Octadecanyl-sulfate | 3 | 4.7 ± 1.2 |
| 12-Sulfonoxy-cis-9-octadecenoic | 4 | >100 |
| acid | | |
| 12-Sulfonoxy-trans-9- | 5 | 9.7 ± 1.3 |
| octadecenoic acid | | |
| α-Sulfostearic acid | 10 | 9.6 ± 0.6 |
| Sodium dodecyl-sulfate | 11 | 5.2 ± 0.6 |
| Sodium dodecyl-sulfonate | 12 | 3.7 ± 0.5 |
| 4-Nitrophenyl sulfate | 13 | >100 |
| Taurolithocholic acid 3-sulfate | 14 | 5 ± 4 |
| Taurocholic acid | 15 | >100 |
| Estrone-3-sulfate | 16 | >100 |
| D-Galactose-6-sulfate | 17 | >100 |
| L-Ascorbic acid 2-sulfate | 18 | >100 |
| dipotassium salt | | |
| N-Acetyl-D-galactosamine 4- | 19 | >100 |
| sulfate | | |

^{a}Results are average ± SD of three separated experiments.

Since phoshonates are also used to inhibit phosphatases (Zabell et al., Bioorg. Med. Chem. 12, 1867-1880 (2004); Cheng, F., and Oldfield, E., J. med. Chem. 47, 5149-5158 (2004)), we investigated the inhibition potency of commercially available phosphonates on Nterm-phos activity (Table 6). Significant inhibition was obtained for three of the phosphonates tested, compounds 27, 32 and 33. The first one is very hydrophobic. Interestingly, the second one, compound 32, is a mimic of farnesyl-pyrophosphate (Cheng, F., and Oldfield, E., J. med. Chem. 47, 5149-5158 (2004)). Compound 33 is structurally similar to 11 and 12, and has a higher IC₅₀ than the sulfur containing compounds (11 and 12), suggesting that sulfonates and sulfates are better inhibitors of Nterm-phos than phosphonates.

TABLE 6
Effect of phosphonates on Human sEH Nterm-Phos activity.

| Name | N^{o}. | IC₅₀ (μM)^{a} |
|---|---|---|
| Tetra-isopropyl-methylene | 20 | >100 |
| diphosphonate | | |
| Diethyl-vinylphosphonate | 21 | >100 |
| Diethyl-benzoylphosphonate | 22 | >100 |
| Diethyl cyclopropyl methyl- | 23 | >100 |
| phosphonate | | |
| Diethyl trans-cinnamyl- | 24 | >100 |
| phosphonate | | |
| Diethyl 4-methylbenzyl- | 25 | >100 |
| phosphonate | | |
| Diethyl allyl-phosphonate | 26 | >100 |
| Dioctyl-phenyl-phosphonate | 27 | 13 ± 1 |
| Di-benzyl-phosphate | 28 | >100 |
| Dimethyl (2-oxoheptyl)- | 29 | >100 |
| phosphonate | | |
| Diethyl (2,2,2-trifluoro-1- | 30 | >100 |
| hydroxyethyl)-phosphonate | | |
| Diethyl (ethyltiomethyl)phosphonate | 31 | >100 |
| α-Hydroxyfarnesylphosphonic | 32 | 73 ± 5 |
| acid | | |
| Dodecyl phosphonic acid | 33 | 40 ± 4 |

^{a}Results are average ± SD of three separated experiments.

To verify that the observed inhibition is not an artifact, we tested the effect of a 10-fold increase in the BSA concentration in the buffer on the inhibition potency. No change in IC₅₀ values was observed, suggesting that these inhibitors do not act by forming non-specific aggregates with the enzyme (McGovern et al., J. Med. Chem. 45, 1712-1722 (2002)). Because some of the compounds tested, such as compound 11, are used as detergents, one could suggest that the observed inhibition is due to a surfactant effect. However, the range of IC₅₀s observed (3 to 100 μM) is far lower than the critical micelle concentrations of these compounds which is generally in the low to mid-millimolar range (Granjeiro et al., Mol. Cell. Biochem. 265, 133-140 (2004)), suggesting that the observed effect is not due to a detergent effect. To test the specificity of the inhibitors for Nterm-phos, we tested the inhibition of alkaline phosphatase from human placenta by compounds 1 to 5 and 10 to 33. No significant inhibition was obtained for any compounds at 100 μM (results not shown).

### Example 6

### Mechanism of Inhibition.

To understand the mode of action of these new inhibitors, compounds 1 and 2 were tested as substrates for the Nterm-phos. Enzyme was incubated (400 nM) with the compounds (100 μM) for an hour and analyzed the mixture by LC-MS to detect any alcohol or diol formed (Newman et al., Proc. Natl. Acad. Sci. USA. 100, 1558-1563 (2003)). Formation of any alcohol or diol was not detected. IC₅₀s were determined for compounds 1-5, 11, 12 and 14 for several incubation times (0, 5, 15 and 30 min) with the enzyme before addition of the substrate; no changes in IC₅₀s were observed. These results support the fact that sulfates are not substrates for Nterm-phos, which was previously demonstrated for compound 13 (Cronin et al., Proc. Natl. Acad. Sci. USA 100, 1552-1557 (2003)).

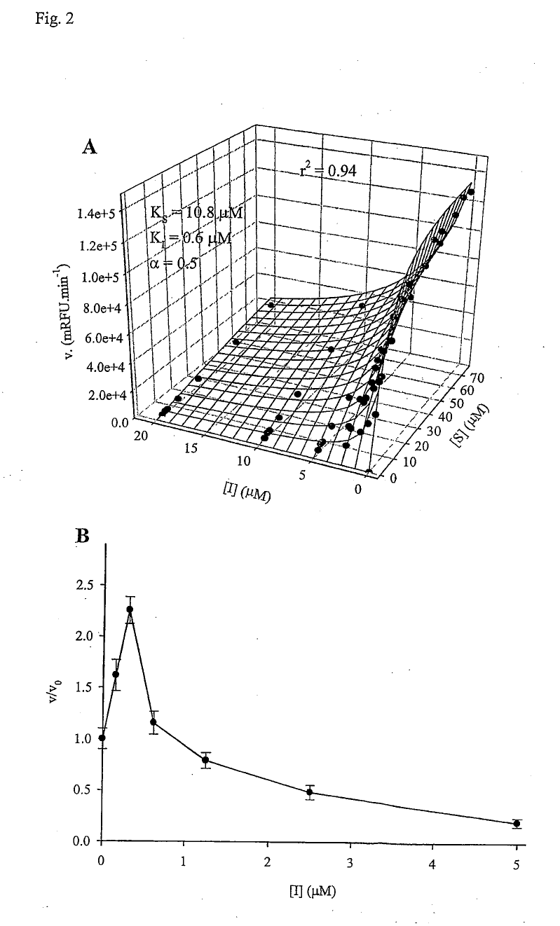

The kinetic constant for compound 1 were determined. A variety of kinetic models were evaluated using Sigma Plot. The simple and mixed-type inhibition models fit poorly to our data (r²<0.4). For each inhibitor concentrations, we obtained sigmoidal velocity curves which suggest an allosteric inhibition model, and we obtained similar V_{M} results which suggest a competitive inhibition model, and in fact the data was best fitted with an allosteric competitive inhibition model. This mechanism of inhibition for an enzyme with two equivalent active sites is described by the following equilibrium (Segel, I. H., Enzyme kinetics: behavior and analysis of rapid equilibrium and steady state enzyme systems, Wiley, New York (1993)).

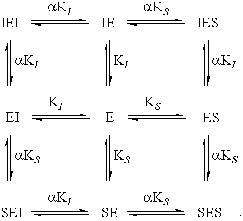

Where the inhibitor, I, could bind at the same sites that the substrate, S, can bind the enzyme E. The binding of both the substrate and inhibitor changes the dissociation constant of the remaining vacant site for I or S by a factor α. The velocity for this type of inhibition is given by equation 1 (Segel, I. H., Enzyme kinetics: behavior and analysis of rapid equilibrium and steady state enzyme systems, Wiley, New York (1993)). To determine K_{S}, K_{I} and α, the velocity (v) results obtained for various concentration of I and S were fitted to Eq. 1 using the value of V_{M} obtained in the absence of inhibitor. A typical result is shown in FIG. 2A. We obtained an average (n=3) K_{S} of 9.8±0.7 μM, an average K_{I} of 0.7±0.3 μM and a factor α of 0.4±0.1 and r² above 0.91. Analysis of the curve fitting revealed that the residual variation was mainly observed for [I]=μM; at this concentration we obtained twice as much inhibition than predicted, and calculated values are significantly different p<0.01) from obtained values. Removing the data for this concentration of inhibitor resulted in a significantly better fit of the model (r² above 0.96), while obtaining similar kinetic parameter values (K_{S}=9.5±0.8 μM, K_{I}=0.6±0.2 μM and α=0.5±0.1 (n =3)). Other allosteric inhibition models did not fit as well (r²<0.8). For the competitive allosteric inhibition model described herein, the inhibitor can also act as an activator (Tricot et al., J. Mol. Biol. 283, 695-704 (1998)). At a low substrate concentration (1 μM), we observed (FIG. 2B) that low concentrations of compound 1 (<0.5 μM) increased the activity of the enzyme while an inhibitory effect was observed for higher concentrations of 1. These results suggest a homotropic cooperativity in the binding of 1, and that the inhibitors described herein bind at the Nterm-phos active site in a manner similar to substrate binding. Interestingly, the K_{S} determined for Attophos® is around 3-fold higher than its observed Kₘ (Table 2), suggesting that the rate of Nterm-phos dephosphorylation is the limiting step for the hydrolysis of this substrate (Fersht, A., Enzyme structure and mechanism, 2^{nd} Edition, W. H. Freeman & Company, New York (1985)).

### Example 7

### Nterm-Phos Endogenous Substrate.

In a previous study, we reported that Nterm-phos prefers lipid phosphates as substrates (Newman et al., Proc. Natl. Acad. Sci. USA. 100, 1558-1563 (2003)). Based on the general inhibitor structure described herein, one could hypothesize that Nterm-phos endogenous substrates are terminal phospho-lipids such as polyisoprenyl phosphates which are important cellular signaling molecules (Levy, B. D., and Serhan, C. N., Biochem. Biophys. Res. Commun. 275, 739-745 (2000)). This is supported by the fact that compound 32, a farnesyl pyrophosphate mimic (Cheng, F., and Oldfield, E., J. med. Chem. 47, 5149-5158 (2004)), is an inhibitor of Nterm-phos. To test this hypothesis, we assayed three isoprenyl pyrophosphates (compounds Segel, I. H., Enzyme kinetics: behavior and analysis of rapid equilibrium and steady state enzyme systems, Wiley, New York (1993); Zabell et al., Bioorg. Med. Chem. 12, 1867-1880 (2004); Cheng, F., and Oldfield, E., J. med. Chem. 47, 5149-5158 (2004)) that are important intermediates in the synthesis of sterols (Levy, B. D., and Serhan, C. N., Biochem. Biophys. Res. Commun. 275, 739-745 (2000)). Interestingly these compounds are substrates for the sEH phosphatase (Table 3). For compound 34, we obtained a linear curve, indicating that the Kₘ for this substrate is above the highest concentration tested (5 μM); and the relatively high V_{M}/Kₘ ratio suggests a high maximal velocity. Increasing the size of the terpene tail, as in compounds 35 & 36, resulted in a lower Kₘ and a slower V_{M}. Compounds 34 to 36 are hydrolyzed by an order of magnitude slower than the previously reported substrate (compound 6 in Table 4).

Because we detected only the alcohol formed (see materials and methods), the kinetic parameters obtained could either result from the direct removal of pyrophosphate or of two phosphates successively with the formation of monophosphate as an intermediate. Concentrating on farnesyl pyrophosphate 35, over a 60 minute incubation time (up to 80% hydrolysis of 35 by human sEH), we were not able to detect farnsyl monophosphate 37 by HPLC with ESI and tandem mass spectrometric detection. Estimated limit of detection for compound 37 was 0.1 μM (signal-to-noise ration equal 3). We were able to detect 0.02 μM pyrophosphate in incubations of sEH with FPP, however its concentration does not change with time of incubation, and it is present in buffer control which suggests that the detected pyrophosphate is an impurity in FPP standard. The kinetic parameters of 37 (Table 7) show that this compound is an excellent substrate for Nterm-phos. It is hydrolyzed approximately 50-fold faster than the corresponding pyrophosphate derivative 34. These results strongly suggest that Nterm-phos is a monophosphatase that hydrolyzes isoprenyl pyrophosphates to the corresponding alcohols and two phosphates molecules. It performs this reaction in two successive steps of phosphate removal with the second being much faster than the first one. Therefore, the kinetic parameters determined for the isoprenyl pyrophosphates (Table 7 compounds 34 to 36) most likely represent the removal of the first phosphate molecule, which is the rate limiting step. Interestingly, compound 37 is hydrolyzed as fast as the previously reported substrate (compound 6 in Table 4).

TABLE 7
Kinetic parameters of Human Nterm-phos for poly-isoprenyl phosphoates.

| | | | V_{M} | | V_{M}/Kₘ |
|---|---|---|---|---|---|
| | | Kₘ | (nmol · min⁻¹ · | Hill | (nmol · min⁻¹ · |
| Name | No. | (μM) | mg⁻¹) | coefficient | mg⁻¹ · μM⁻¹) |
| Geranyl-pyrophosphate | 34 | — | — | — | 5.3 ± 0.3 |
| Farnesyl- | 35 | 10.1 ± 1.1 | 12.5 ± 1.0 | 1.0 ± 0.1 | 1.2 ± 0.2 |
| pyrophosphate | | | | | |
| Geranylgeranyl- | 36 | 3.4 ± 1.6 | 4.7 ± 1.1 | 1.0 ± 0.2 | 1.4 ± 0.8 |
| pyrophosphate | | | | | |
| Farnesyl | 37 | 5.7 ± 0.4 | 303 ± 19 | 1.0 ± 0.1 | 53 ± 5 |
| monophosphate | | | | | |

Results are average ± SD of three separated experiments.

### Example 8

### C-Term EH Inhibition.

We then tested the effect of the Nterm-phos inhibitors on the Cterm-EH activity. As shown on Table 8, significant inhibition was obtained only for a small number of sulfates (1-3, 5 and 11). A slight inhibition was observed for the phosphonate 27. Interestingly, the pattern of inhibition of Cterm-EH is different than the one observed for Nterm-phos. For example, compound 12 is a far better inhibitor of Nterm-phos than compound 11, but it is not an inhibitor of Cterm-EH while compound 11 is. As observed above, increasing the concentration of BSA in the buffer by an order of magnitude does not alter the potency of the inhibitors, suggesting that these inhibitors do not act by forming non-specific aggregates with the enzyme (McGovern et al., J. Med. Chem. 45, 1712-1722 (2002)). Thus, sulfates represent a new class of inhibitors for Cterm-EH activity; previous potent inhibitors described include ureas, amides and carbamates (Morisseau et al., Proc. Natl. Acad. Sci. USA 96, 8849-8854 (1999)).

TABLE 8
Effect of sulfates, pyrophosphates and phosphonates on
Human sEH Cterm-EH activity.

| N^{o}. | IC₅₀ (μM)^{a} |
|---|---|
| 1 | 28 ± 2 |
| 2 | 90 ± 5 |
| 3 | 21 ± 5 |
| 4 | >100 |
| 5 | 16 ± 3 |
| 10 | >100 |
| 11 | 50 ± 5 |
| 12 | >100 |
| 13 | >100 |
| 14 | 90 ± 5 |
| 15 | >100 |
| 16 | >100 |
| 17 | >100 |
| 18 | >100 |
| 19 | >100 |
| 20 | >100 |
| 21 | >100 |
| 22 | >100 |
| 23 | >100 |
| 24 | >100 |
| 25 | >100 |
| 26 | >100 |
| 27 | 92 ± 3 |
| 28 | >100 |
| 29 | >100 |
| 30 | >100 |
| 31 | >100 |
| 32 | >100 |
| 33 | >100 |

^{a}Results are average ± SD of three separated experiments.

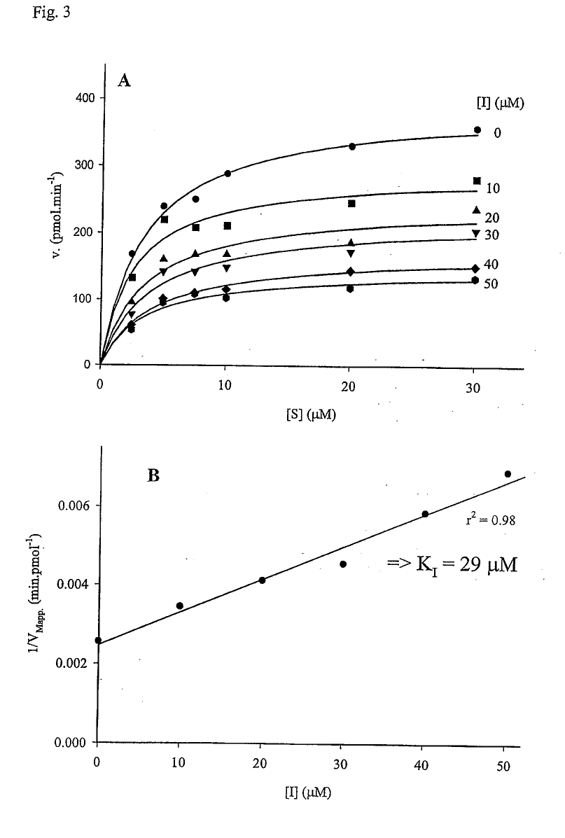

In order to understand the mode of action of these compounds, we determined kinetic constants for compound 1. The best fit was obtained for a non-competitive inhibition mechanism (FIG. 3). This result suggests that the inhibitor does not bind at the active site, or least not exclusively at the active site, as previously described inhibitors do (Morisseau et al., Proc. Natl. Acad. Sci. USA 96, 8849-8854 (1999)), thus showing that these new inhibitors of Cterm-EH act at a different site on the enzyme. We found for compound 1 at the Cterm-EH a K_{I} of 31±2 μM (n=3), which is roughly 100-fold the K_{I} obtained for this compound at the Nterm-Phos (see above). This result suggests that the inhibition of Cterm-EH by compound 1 does not come from its binding to the pocket of Nterm-Phos. To confirm this hypothesis, we tested the effect of compounds 1-5 on the EH activity of the full-length and N-terminally truncated (with only the C-terminus present) human sEH, and the cress sEH which does not contain a mammalian-like N-terminal domain (Beetham et al., DNA Cell Biol. 14, 61-71 (1995)). Similar patterns of inhibition (Table 9) were obtained for both full-length and truncated human sEH, while no inhibition was observed for the plant sEH. The small differences observed between the full length and truncated human sEH are probably linked to the use of a truncated enzyme from crude extract versus purified full-length human sEH. These results suggest that sulfates inhibit Cterm-EH by binding to a site on the C-terminal domain that is distinct from the EH active site, and this site is not present on the plant sEH.

TABLE 9
effect of 100 μM of lipid sulfates on the EH activity of the full length and
truncated human sEH, and cress sEH.

Human sEH

| | Full length | Truncated | Cress sEH |
|---|---|---|---|
| N^{o}. | Inhibition at 100 μM (%)^{a} | | |
| 1 | 86 ± 2 | 89 ± 3 | <2 |
| 2 | 54 ± 3 | 58 ± 2 | <2 |
| 3 | 72 ± 2 | 67 ± 3 | <2 |
| 4 | 26 ± 3 | 16 ± 2 | <2 |
| 5 | 68 ± 2 | 44 ± 4 | <2 |

^{a}Results are average ± SD of three separated experiments.

In order to understand the mode of action of these compounds, we determined kinetic constants for compound 1. The best fit was obtained for a non-competitive inhibition mechanism (FIG. 3). This result suggests that the inhibitor does not bind at the active site, or least not exclusively at the active site, as previously described inhibitors do (Morisseau et al., Proc. Natl. Acad. Sci. USA 96, 8849-8854 (1999)), thus showing that these new inhibitors of Cterm-EH act at a different site on the enzyme. We found for compound 1 at the Cterm-EH a K_{I} of 31±2 μM (n=3), which is roughly 100-fold the K_{I} obtained for this compound at the Nterm-Phos (see above). This result suggests that the inhibition of Cterm-EH by compound 1 does not come from its binding to the pocket of Nterm-Phos. To confirm this hypothesis, we tested the effect of compounds 1-5 on the EH activity of the full-length and N-terminally truncated (with only the C-terminus present) human sEH, and the cress sEH which does not contain a mammalian-like N-terminal domain (Beetham et al., DNA Cell Biol. 14, 61-71 (1995)). Similar patterns of inhibition (Table 7) were obtained for both full-length and truncated human sEH, while no inhibition was observed for the plant sEH. The small differences observed between the full length and truncated human sEH are probably linked to the use of a truncated enzyme from crude extract versus purified full-length human sEH. These results suggest that sulfates inhibit Cterm-EH by binding to a site on the C-terminal domain that is distinct from the EH active site, and this site is not present on the plant sEH.

The recent discovery of the Nterm-phos activity (Cronin et al., Proc. Natl. Acad. Sci. USA 100, 1552-1557 (2003); Newman et al., Proc. Natl. Acad. Sci. USA. 100, 1558-1563 (2003)) has revealed a gap in our knowledge about the functional role of this enzyme. To fill this gap, new tools are needed. We report herein the use of Attophos®, compound 9, as a new surrogate substrate for this activity; not only does it have a Kₛ in the low μM range, it also displays a positive cooperative binding similar to compound 1 (Newman et al., Proc. Natl. Acad. Sci. USA. 100, 1558-1563 (2003)). Compared to the assay using the latter substrate, use of Attophos® gives an assay that is more sensitive and easier to perform. Furthermore, we were able to execute the fluorescent assay in a 96-well format, permitting us to quickly screen chemicals for Nterm-phos inhibition.

Using this new assay format, we investigated the effect of several pharmacophores on the inhibition of the sEH phosphatase activity. The results clearly show that sulfates, sulfonates and phosphonates represent a new class of potent inhibitors of the Nterm-phos activity of sEH. Moreover, the inhibition is enhanced by the presence of a hydrophobic linear or cyclic tail; the presence of a carboxylic function or a double bond reduced the inhibition potency only slightly, except for the presence of a cis double bond. While surprising, this latter result was confirmed by testing compound 4 from several synthetic batches and from commercial sources (City Chemicals). The inhibition caused by these compounds does not decrease over time. One of the more potent inhibitors tested, compound 1, has a high nanomolar K_{I} that is roughly 20-fold the enzyme concentration tested and 10-fold lower than the K_{S} of the substrate, indicating that this compound binds relatively tightly to the enzyme. One could envision that optimization of the structure will yield stochiometric inhibitors of Nterm-phos activity. The exact mechanism by which the sulfates, sulfonates and phosphonates inhibit the Nterm-phos is not known. The kinetic inhibition was best described by a competitive model for which the inhibitor has a positive allosteric effect, like that observed for the substrate. This strongly suggests that the inhibitors mimic the binding of the substrate to the active site (FIG. 4). The inhibitors most likely establish hydrogen bonds between their hydrophilic heads and residues within the active site. Furthermore, the hydrophobic tail of the inhibitors most likely bind through Van der Waals interactions to a ˜14 Å long hydrophobic tunnel with one end at the Nterm-phos active site and the other end near the interface of the N- and C-terminal domains (Gomez et al., Biochemistry 43, 4716-4723 (2004)). It is not known which part of the inhibitor or substrate binding is responsible for the observed homotrophic cooperativity. Clearly, future structure determination and site-directed mutagenesis experiments are required to probe the allosteric regulation of Nterm-phos.

Due to the allosteric effects observed for Nterm-phos and the fact that the two N-terminal domains of each homodimer do not form contacts with one another (Argiriadi et al., Proc. Natl. Acad. Sci. USA 96, 10637-10642 (1999); Gomez et al., Biochemistry 43, 4716-4723 (2004)), we believe that binding at the N-terminal active site could influence the Cterm-EH activity. While we found that some of the N-terminal inhibitors did affect the C-terminal activity, the results obtained clearly show that this effect is not through binding at the N-terminal. The data suggest the presence of a new binding site on the C-terminal domain that is distinct from the Cterm-EH catalytic site. The future discovery of inhibitors that binds exclusively to this latter site will be a valuable tool to probe the role of this site in the in vivo regulation of epoxide hydrolysis by sEH, which is an important process for blood pressure and inflammation regulation (Newman et al., Prog. Lipid Res. 44, 1-51 (2005)).

The mammalian soluble epoxide hydrolase is a unique enzyme in that it has the uncommon characteristic of having two enzymatic activities. While the role of the Cterm-EH activity in inflammation and hypertension, via epoxy fatty acid hydrolysis, is well documented (Newman et al., Prog. Lipid Res. 44, 1-51 (2005)), the role of the Nterm-phos remains to be elucidated. In a previous study, we reported that Nterm-phos prefers lipid phosphates as substrates (Newman et al., Proc. Natl. Acad. Sci. USA. 100, 1558-1563 (2003)). Based on the general inhibitor structure described herein, we found that poly-isoprenyl phosphates are also good substrates for Nterm-phos. Polyisoprenyl phosphates are important cellular signaling molecules, thus suggesting a possible role for Nterm-phos in sterol synthesis or inflammation (Levy, B. D., and Serhan, C. N., Biochem. Biophys. Res. Commun. 275, 739-745 (2000); Holstein, S. A., and Hohl, R. J., Lipids 39, 293-309 (2004)). Alternatively, since a sterol sulfate, compound 14, inhibits the enzyme, sterol phosphates may also be substrates for Nterm-phos. Ultimately, the inhibitors developed and described herein provide valuable tools to investigate the biological role of the Nterm-phos.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for inhibiting epoxide hydrolase (EH), comprising contacting said soluble epoxide hydrolase with an inhibiting amount of a compound having the structure: 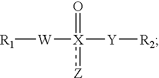
wherein
W is selected from the group consisting of a NH, O, S and CHₙ;
X is selected from the group consisting of As, N, P, Se and S;
Y is selected from the group consisting of NH, O, S and CHₙ;
Z is selected from -the group consisting of N, O and S, or Z can be absent;
n is 0, 1, 2 or 3;
R₁ is selected from the group consisting of C₁-C₈alkyl, C₂-C₆ alkenyl, C₂-C₆ alkynyl, heteroC₁-C₈alkyl, C₃-C₁₂cycloalky, aryl and heterocyclyl;
and
R₂ is selected from the group consisting of H, C₁-C₈alkyl, C₂-C₆alkenyl, C₂--C₆alkynyl, heteroC₁-C₈alkyl, C₃-C₁₂cycloalky, aryl and heterocyclyl; wherein each R₁ and R₂ is optionally, independently substituted with from 1 to 6 R₃ substituents selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
the dashed line indicates an optional double bond; and pharmaceutically derivatives thereof.

2. A method of claim 1 wherein the compound is inhibiting the phosphatase activity of said epoxide hydrolase.

3. A method for maintaining the concentration of a biologically active phosphate, said method comprising contacting said soluble epoxide hydrolase with an amount of an inhibitor of the phosphatase activity of said epoxide hydrolase.

4. A method of increasing sodium excretion in a subject, said method comprising administering to said subject an effective amount of an inhibitor of the phosphatase activity of epoxide hydrolase.

5. A method of regulating endothelial cell function in a subject, said method comprising administering to said subject an effective amount of an inhibitor of the phosphatase activity of epoxide hydrolase.

6. A method of treating a disease modulated by soluble epoxide hydrolase, said method comprising administering to the patient a therapeutically effective amount of an inhibitor of the phosphatase activity of epoxide hydrolase.

7. A method in accordance with claim 6, wherein said disease is selected from the group consisting of hypertension, inflammation, adult respiratory distress syndrome; diabetes or its complications; end stage renal disease; Raynaud syndrome, arthritis, erectile dysfunction, renal deterioration, nephropathy, high blood pressure, obstructive pulmonary disease, interstitial lung disease and asthma.

8. The method in accordance with claim 7, wherein said disease is inflammation.

9. The method in accordance with claim 8, wherein said inflammation is selected from the group consisting of renal inflammation, vascular inflammation, lung inflammation, endothelial cell inflammation.

10. A method of any one of claims 1 to 9, wherein the inhibitor is complementary to a portion of the phosphatase active site of epoxide hydrolase.

11. A method of claim 10, wherein the inhibitor has the structure: 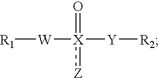
wherein
W is selected from the group consisting of a NH, O, S and CHₙ;
X is selected from the group consisting of As, N, P, Se and S;
Y is selected from the group consisting of NH, O, S and CHₙ;
Z is selected from the group consisting of N, O and S, or Z can be absent;
n is 0, 1, 2 or 3;
R₁ is selected from the group consisting of C₁-C₈alkyl, C₂-C₆ alkenyl, C₂-C₆ alkynyl, heteroC₁-C₈alkyl, C₃-C₁₂cycloalky, aryl and heterocyclyl;
and
R₂ is selected from the group consisting of H, C₁-C₈alkyl, C₂-C₆alkenyl, C₂-C₆alkynyl, heteroC₁-C₈alkyl, C₃-C₁₂cycloalky, aryl and heterocyclyl; wherein each R₁ and R₂ is optionally, independently substituted with from 1 to 6 R₃ substituents selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
the dashed line indicates an optional double bond; and pharmaceutically derivatives thereof.

12. A method of claim 11, wherein W is NH.

13. A method of claim 11, wherein W is O.

14. A method of claim 11, wherein W is S.

15. A method of claim 11, wherein W is CHₙ.

16. A method of claim 11, wherein W is NH.

17. A method of claim 11, wherein W is O.

18. A method of claim 11, wherein W is S.

19. A method of claim 11, wherein W is CHₙ.

20. A method of claim 11, wherein Y is NH.

21. A method of claim 11, wherein Y is O.

22. A method of claim 11, wherein Y is S.

23. A method of claim 11, wherein Y is CHₙ.

24. A method of claim 11, wherein Z is N.

25. A method of claim 11, wherein Z is O.

26. A method of claim 11, wherein Z is S.

27. A method of claim 11, wherein Z is absent.

28. A method of claim 1, wherein W, Y and Z is O; and X is S.

29. A method of claim 11, wherein n is 1.

30. A method of claim 11, wherein n is 2.

31. A method of claim 11, wherein n is 3.

32. A method of claim 11, wherein R₁ is alkyl.

33. A method of claim 11, wherein R₁ is cycloalkyl.

34. A method of claim 11, wherein R₁ is aryl.

35. A method of claim 11, wherein R₁ is heterocyclyl.

36. A method of claim 11, wherein R₂ is alkyl.

37. A method of claim 11, wherein R₂ is cycloalkyl.

38. A method of claim 11, wherein R₂ is aryl.

39. A method of claim 11, wherein R₂ is heterocyclyl.

40. A method of claim 11, wherein R₁ is alkyl.

41. A method of claim 11, wherein R₂ is hydrogen.

42. A method of claim 11, wherein W, Y and Z is O; X is S; R₁ is alkyl; and R₂ is hydrogen.

43. A method of claim 11, wherein the inhibitor has the structure: 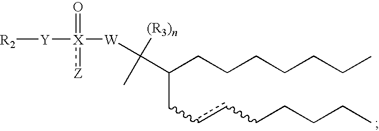
wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

44. A method of claim 45, wherein R₃ is selected from the group consisting of C₁-C₈alkyl, hydroxyl, carboxy and C₁-C₈alkylcarboxy.

45. A method of claim 11, wherein the inhibitor has the structure: 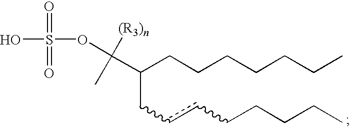
wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

46. A method of claim 45, wherein R₃ is selected from the group consisting of C₁-C₈alkyl, hydroxyl, carboxy and C₁-C₈alkylcarboxy.

47. A method of claim 45, having a structure selected from the group consisting of: 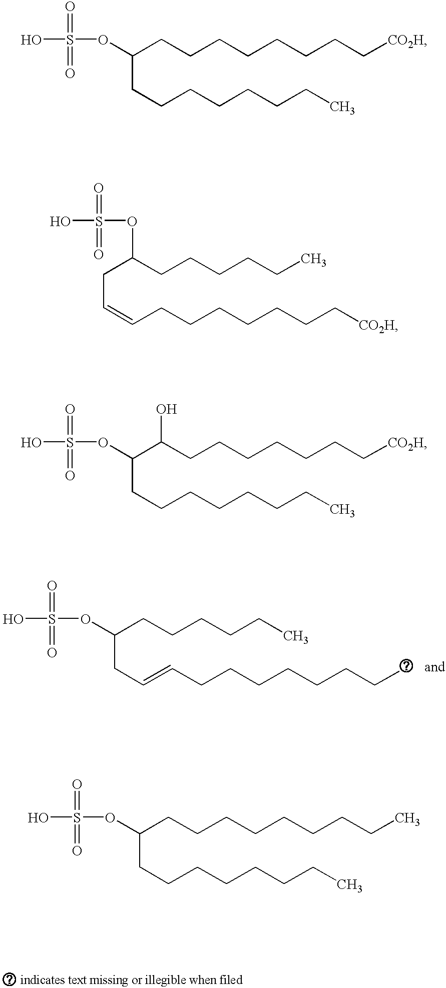
and pharmaceutically acceptable derivatives thereof.

48. A compound having the structure: 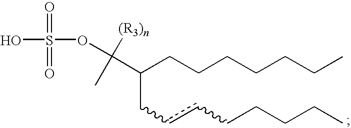
wherein R₃ is selected from the group consisting of halo, nitro, oxo, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxy C₁-C₈alkoxy, haloC₁-C₈alkoxy, thio C₁-C₈alkyl, aryl, aryloxy, C₃-C₈cycloalkyl, C₃-C₈cycloalkyl C₁-C₈alkyl, aryl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₂-C₈alk(en)(yn)yl groups, cyano, formyl, C₁-C₈alkylcarbonyl, arylcarbonyl heteroarylcarbonyl, carboxy, C₁-C₈alkylcarboxy, C₁-C₈alkoxycarbonyl, aryloxycarbonyl, aminocarbonyl, C₁-C₈alkylaminocarbonyl, C₁-C₈dialkylaminocarbonyl, arylaminocarbonyl, diarylaminocarbonyl, arylC₁-C₈alkylaminocarbonyl, aryloxy, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, C₁-C₈alkylarylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl;
n is 0, 1, 2, 3, 4, 5 or 6; the dashed line indicates an optional bond; the wavy line indicates E or Z stereochemistry; and pharmaceutically acceptable derivatives thereof.

49. A compound of claim 48, wherein R₃ is selected from the group consisting of C₁-C₈alkyl, hydroxyl, carboxy and C₁-C₈alkylcarboxy.

50. A compound of claim 48, having a structure selected from the group consisting of: 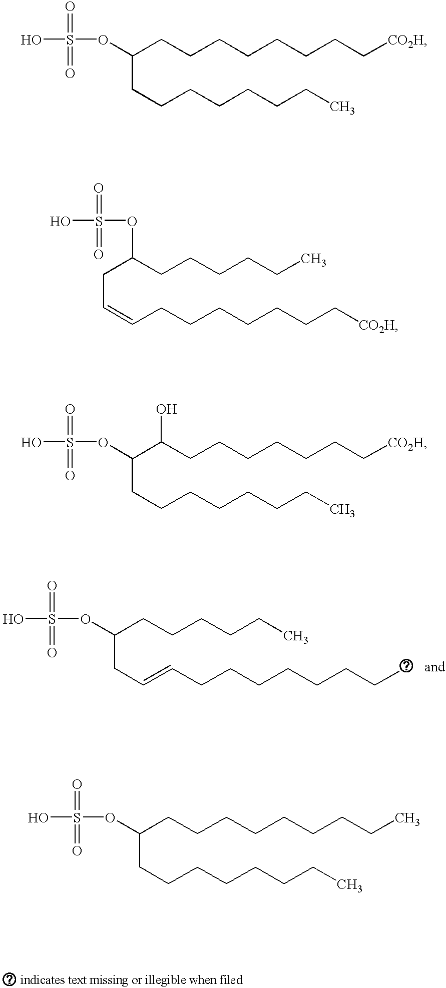
and pharmaceutically acceptable derivatives thereof.

51. A composition comprising an amount of a compound of claim 48, effective to inhibit or decrease phosphatase activity of sEH.

52. Use of a compound of claim 48, effective to inhibit or decrease phosphatase activity of sEH effective for the preparation of a medicament for treating a condition in a mammal which is ameliorated by decreasing or inhibiting the phosphatase activity of sEH.
